(19) United States
(12) Patent Application Publication
(10) Pub. No.: US 2011/0124712 A1
(43) Pub. Date: May 26, 2011

(54) ANTI-CANCER COMPOSITION COMPRISING MICRORNA MOLECULES

(75) Inventors: Jong Bae Park, Seoul (KR); Seung Hoon Lee, Seoul (KR); Eun Kyoung Park, Goyang-si (KR); Donghee Lee, Goyang-si (KR); Hee-Seok Yang, Seoul (KR); Heon Yoo, Seoul (KR); Hea Jin Kim, Goyang-si (KR); Tae Hoon Kim, Seoul (KR); Hee Jin Kwak, Goyang-si (KR)

(73) Assignee: NATIONAL CANCER CENTER, Goyang-si (KR)

(21) Appl. No.: 12/599,733

(22) PCT Filed: Jul. 25, 2008
(86) PCT No.: PCT/KR2008/004377

§ 371 Date: Feb. 1, 2011

(30) Foreign Application Priority Data
Jul. 18, 2008 (KR) 10-2008-0070087

(51) Int. Cl. A61K 31/7105; C07H 21/02; C12N 15/63; C40B 40/06; A61P 35/00; A61P 9/10; A61P 9/00; A61P 17/00; A61P 27/06; A61P 27/02; A61P 19/02; A61P 17/06; A61P 25/28

(52) U.S. Cl. 514/44 R; 536/23.1; 435/320.1; 506/16

(57) ABSTRACT

Disclosed is an anticancer composition for the treatment of hypoxia-induced angiogenesis-associated diseases including cancers. It comprises a microRNA-125 nucleic acid molecule. Also, methods of inhibiting angiogenesis, suppressing the invasion and metastasis of cancer cells, and treating cancers are provided.

### TECHNICAL FIELD

The present invention relates to novel uses of microRNA125 (mir-125). More particularly, the present invention relates to a composition for the treatment of hypoxia-induced angiogenesis-associated diseases including cancer. Also, the present invention is concerned with methods for inhibiting angiogenesis and suppressing the invasion and metastasis of cancer cells characterized by a low expression level of microRNA-125.

### BACKGROUND ART

MicroRNAs are a newly discovered class of tiny regulatory molecules which appear to control many biological processes within cells. They are encoded for by genomes in animal and plant cells and bind to mRNAs to regulate the expression of the corresponding genes (He and Harmon, 2004). There are growing evidences that microRNAs are also associated with the onset and progression of many diseases including cancer, viral infection, heart diseases, neuropathy, etc. Therefore, there have been many efforts to use microRNAs in clinical applications. For example, based on the disclosure that microRNA-122 stimulates hepatitis virus (Science 309, 1577-1781; 2005), Santaris Pharma has recently developed a locked nucleic acid (LNA), an antagonist of the liver-specific microRNA-122, which is known to reduce blood cholesterol levels and block Hepatitis C virus (HCV) (Nature 452, 896-900; 2008).

In general, cancer cells proliferate faster than endothelial cells, which form the linings of blood vessels. As cancer cells grow quickly, the newly formed blood vessels of the tumor tissue are insufficiently distributed therein and thus the tumor tissue cannot be supplied with sufficient blood. This insufficiency of blood supply to cancer cells induces nutrient and oxygen deficiencies in and acidification of the tumor tissue. In fact, partial oxygen pressures are measured to have a median value of from 40 to 60 mmHg in normal tissue, but a median value of 10 mmHg or less for the most part in solid cancer (Brown J M, Cancer Res 59, 5863-5870, 1999). Under such hypoxic conditions inside cancer tissue, cancer cells have been found to produce proteins used to supply nutrients and oxygen necessary for their survival and proliferation. In this context, hypoxia-inducible proteins include vascular endothelial growth factor (VEGF), EPO (erythropoietin), glycolytic enzymes, etc.

Accordingly, the regulation of VEGF, a representative hormonal protein responsible for the angiogenesis of cancer cells, is likely to lead to the inhibition of cancer metastasis. Particularly for solid cancers, it is known that chemical therapy or radiotherapy is not effective as concerns the hypoxic inside of the tumor. Thus, there is a need for gene therapy that targets a specific regulatory factor.

In addition, it is important in cancer treatment to prevent cancer invasion and metastasis into other tissues as well as to inhibit the growth of the cancer itself. Particularly, most cases of brain cancer re-occur as the result of invasion and surgery is ineffective for the treatment thereof, and they are limitedly treated with chemical therapy or radiation therapy. Hence, the inhibition of invasion is indispensible for the treatment of brain cancer.

If developed, a genetic factor capable of regulating angiogenesis in cancer cells under hypoxic conditions could be used as a therapeutic effectiveness in the treatment of various cancers which are difficult because of the prognosis to treat with surgery, chemical therapy and radiation therapy.

Leading to the present invention, intensive and thorough research into anti-angiogenic factors active in hypoxic conditions, conducted by the present inventors using a microarray technique for assaying microRNAs between normal cells and various cancer cells in hypoxic conditions, resulted in the finding that the expression of microRNA-125 is specifically reduced in hypoxic cancer cells and serves as an anti-angiogenic factor by regulating VEGF secretion in a state of hypoxia.

### DISCLOSURE

### Technical Problem

It is therefore an object of the present invention to provide an anticancer composition comprising a microRNA-125 nucleic acid molecule.

It is another object of the present invention to provide a composition for the treatment of hypoxia-induced angiogenesis-associated diseases, comprising a microRNA-125 nucleic acid.

It is a further object of the present invention to provide a marker composition for the diagnosis of brain cancer and brain tumors, comprising an agent for measuring a microRNA-125 expression level.

It is still a further object of the present invention to provide a method for inhibiting hypoxia-induced angiogenesis, comprising using the microRNA-125 nucleic acid molecule.

It is still another object of the present invention to provide a method for the suppression of the invasion and metastasis of cancer cells, comprising using the microRNA-125 nucleic acid molecule.

It is yet another object of the present invention to provide a method for the treatment of hypoxia-induced angiogenesis-associated diseases, particularly, cancers, comprising using the microRNA-125 nucleic acid molecule.

### Technical Solution

In accordance with an aspect thereof, the present invention pertains to an anticancer composition comprising a microRNA-125 nucleic acid molecule.

As used herein, the term “microRNA-125 nucleic acid molecule” means a single- or double-stranded nucleic acid molecule constituting microRNA-125. Throughout the specification, ‘microRNA-125’ is interchangeably used with ‘mir-125’.

microRNAs are, for the most part, encoded by introns on chromosomes and transcribed as primary transcripts which are then processed to shorter structures, known as precursor microRNAs (pre-miRNAs), by Drosha in the cell nucleus. Following nuclear export under the mediation of exportin, these pre-miRNAs are further processed to mature, about 22 bp-long miRNAs in the cytoplasm by interaction with Dicer which are associated with RNA interference silencing complex (RISC) to do gene silencing functions (Nat Rev Mol Cell Biol 6, 376-385; 2005).

microRNA-125 (mir125) is a family of various microRNAs including mir125a, 125b1 and 125b2, sharing the same seed sequence. mir-125 was first discovered as a homolog thereof, known as lin-4, in C. elegans and a mouse homolog was described in 2002 by Lagos-Quintana. As for human mir-125, it was reported in 2005 by Lee et al. that miR125b1 is located at an exon region of a functionally unknown gene on chromosome 11 q24.1 and miR125b2 is located at an intron region of a gene on chromosome 21 q21.1. miR125a was found on chromosome 19 q13.4 in 2007 by Gross et al. (see FIG. 6).

Useful in the present invention are the microRNA-125 homologs derived from human and non-human animals including monkeys, pigs, horses, cows, sheep, dogs, cats, mice, rabbits, and the like. Preferred examples include human microRNA-125a, microRNA-125b1 and microRNA-125b2, but are not limited thereto.

The microRNA-125 nucleic acid molecules according to the present invention may range in length from 18 to 100 nt (nucleotides) and may be mature microRNA-125s with a length of preferably from 19 to 25 nt and more preferably from 21 to 23 nt. Alternatively, the microRNA-125 nucleic acid molecules of the present invention may be provided as precursor microRNA-125 molecules with a length of from 50 to 100 nt and preferably from 65 to 95 nt. The nucleotide sequences of both the mature and the precursor microRNA-125 molecules are publicly available by reference to the database of the National Institute of Health (NIH), GenBank mir125a(406910), mir125b1(406911), mir125b2(406912) and to miRBASE (http://microrna.sanger.ac.uk/), for example, mir125a (Accession No. MI0000469 (ID: hsa-mir-125a) for the precursor form, and Accession Nos. MIMAT0000443 (ID: hsa-miR-125a-5p, SEQ ID NO. 1) and MIMAT0004602 (ID: hsa-miR-125a-3p, SEQ ID NO. 2) for the mature forms), mir125b1 (Accession No. MI0000446 (ID: hsa-mir-125b-1) for the precursor form, and Accession Nos. MIMAT0000423 (ID: hsa-miR-125b, SEQ ID NO. 3) and MIMAT0004592 (ID: hsa-miR-125b-1, SEQ ID NO. 4) for the mature forms), and mir125b2 (Accession No. MI0000470 (ID: hsa-mir-125b-2) for the precursor form, and Accession Nos. MIMAT0000423 (ID: hsa-miR-125b, SEQ ID NO. 3) and MIMAT0004603 (ID: hsa-miR-125b-2, SEQ ID NO. 5) for the mature forms) (FIG. 6). It should be appreciated that the microRNA-125 nucleic acid molecules of the present invention include functional equivalents of the constituent nucleic acid molecules, that is, variants which show the same functions as those of intact microRNA-125 nucleic acid molecules although they are mutated by deletion, substitution or insertion of some nucleotide residues.

Further, the microRNA-125 nucleic acid molecules of the present invention may exist in single-stranded or double stranded forms. Mature microRNA molecules are primarily in single-stranded forms while precursor microRNAs are partially self-complementary to form double-stranded structures (for example, stem-loop structures). In an alternative embodiment, the nucleic acid molecules of the present invention may be in the form of RNA, DNA, PNA (peptide nucleic acid) or LNA (locked nucleic acid).

The nucleic acid molecules of the present invention may be isolated or prepared using standard molecular biology techniques, e. g, chemical synthesis or recombinant technology, or may be commercially available.

In addition to the microRNA-125 (mir-125) nucleic acid molecules, the anticancer composition of the present invention may comprise a material capable of improving the expression of the microRNA-125 in cells, such as synthetic or natural compounds or proteins, etc.

The term “anticancer”, as used herein, is intended to mean inhibitive of the growth of cancer cells, fatal to cancer cells, and/or suppressive or oppressive of the metastasis of cancer cells, in relation to the prevention and treatment of cancer. Herein, the term “prevention of cancer” is intended to refer to any action resulting in the suppression of carcinogenesis or the delay of cancer through the administration of the composition. The term “treatment of cancer”, as used herein, is intended to refer to any action resulting in improvement in cancer symptoms or beneficial alternation of cancer state through the administration of the composition.

Because they inhibit angiogenesis in conditions of hypoxia, the microRNA-125 nucleic acid molecules of the present invention are of anticancer activity. The inhibition of angiogenesis in a state of hypoxia results from the suppression of vascular endothelial growth factor (VEGF) secretion.

As used herein, the term “hypoxic condition” or “hypoxia” is intended to refer to a cellular or tissue oxygen level lower than a physiologically acceptable level, e.g., an optimal oxygen level necessary for normal cell or tissue activity.

Generally, cancer cells proliferate at higher rates than neighboring blood endothelial cells do, and thus are subjected to deficiency in nutrient and oxygen and acidification because they are supplied with insufficient blood. Hence, the tumor tissues express proteins functioning to supply nutrients and oxygen necessary for their survival and proliferation. VEGF is representative of such secreted proteins.

Suppressive of hypoxia-induced VEGF (vascular endothelial growth factor) secretion, the microRNA-125 nucleic acid molecules of the present invention can inhibit VEGF-mediated angiogenesis. Particularly, the microRNA-125 nucleic acid molecules of the present invention suppress the VEGF expression induced by the inactivation of PTEN (phosphatase and tensin homolog deleted on chromosome ten), resulting in the inhibition of angiogenesis.

In addition, the microRNA-125 nucleic acid molecules of the present invention show anticancer activity by suppressing the invasion and metastasis of cancer cells. The term “metastasis of cancer cells”, as used herein, means the migration of cancer cells from a primary tumor to a distal tissue or organ. In metastasis, cancer cells penetrate into adjacent tissues and enter blood vessels, which is generically called invasion. Then, the cancer cells circulate through the bloodstream and settle down to grow within normal tissues elsewhere in the body. Thus, invasion is closely related with metastasis and the migration of cancer cells. The microRNA-125 nucleic acid molecules of the present invention are suppressive particularly of the invasion of cancer cells, thereby preventing cancer cells from metastasizing and proliferation, also.

The anticancer composition according to the present invention is applicable for the treatment of any cancer as long as its incidence is associated with the abnormal expression of microRNA-125, as exemplified by carcinoma, lymphoma, blastoma, sarcoma, and leukemia. Preferably, examples of the cancer to which the anticancer composition of the present invention can be therapeutically applied include brain cancer, cervical cancer, breast cancer, bladder cancer, liver cancer, prostate cancer and neuroblastoma. Particularly, the anticancer composition of the present invention is useful in the prevention and treatment of cancers low in microRNA-125 expression level, and such preferably-treated cancers with low microRNA-125 levels as are listed in Table 1, below, and more preferably brain cancer, but are not limited thereto. As used herein, the term “brain cancer” is intended to mean all cancer tissues occurring in the brain, including brain tumors.

TABLE 1

| miRNA | Cell Line | Regulate | Reference |
|---|---|---|---|
| 125a | Tumor breast tissue | Down | Iorio et al. 2005 |
| | | | (Cancer Res) |
| | Hepatocellular | Down | Murakami et al. |
| | carcinoma | | 2006 (Oncogene) |
| | Prostate carcinoma | Down | Porkka et al. 2007 |
| | | | (Cancer Res) |
| | Pancreas | Up | Volinia et al. |
| | | | 2006 (PNAS) |
| 125b | Head & neck cancer | Up | Tran et al. 2007 |
| | cell line | | (BBRC) |
| | Prostate carcinoma | Down | Porkka et al. 2007 |
| | | | (Cancer Res) |
| | Mouse model for | Up | Van Rooij et al. |
| | cardiac hypertrophy | | 2006 (PNAS) |
| b1&b2 | Tumor breast tissue | Down | Iorio et al. 2005 |
| | | | (Cancer Res) |
| | Breast | Down | Volinia et al. |
| | | | 2006 (PNAS) |
| | Pancreas | Up | Volinia et al. |
| | | | 2006 (PNAS) |
| | Stomach | Up | Volinia et al. |
| | | | 2006 (PNAS) |
| 125a&b | Primary neuroblastoma | Down | Laneve et al. 2007 |
| | tumors | | (PNAS) |

In accordance with another aspect thereof, the present invention pertains to a composition for the treatment of diseases associated with hypoxia-induced angiogenesis, comprising a microRNA-125 nucleic acid molecule.

Having inhibitory activity against angiogenesis by suppressing VEGF secretion in a hypoxic condition, the microRNA-125 nucleic acid molecules of the present invention can be used as therapeutics for diseases associated with hypoxia-induced angiogenesis. Examples of the angiogenesis-associated diseases to which the microRNA-125 nucleic acid molecules of the present invention are applicable include cancer, angioma, angiofibroma, arteriosclerosis, vascular adhesion, scleroderma, neovascular glaucoma, diabetic retinopathy, neovascular corneal disorders, arthritis, psoriasis, telangectasia, pyogenic granuloma, and Alzheimer's disease, but are not limited thereto. Preferably, the composition is applied to lesion tissues with low expression levels of microRNA-125.

In the practice of the present invention, the present inventors analyzed microRNA levels in normal cells and various cancer cells under hypoxic conditions using microarray technology and examined the influence of the decreased level of microRNAs on hypoxia-induced VEGF expression in cancer cells, resulting in the finding that microRNA-125 (mir-125a and mir-125b) reduces VEGF secretion levels in a hypoxic condition (FIGS. 1 and 2). In addition, an examination was made of the cellular mechanism by which the selected microRNA-125 is regulated.

In a hypoxic condition, VEGF secretion was regulated by microRNA-125 or PTEN rather than by HIF-1. Also, when HIF-1 inhibition or PTEN expression was induced in normoxia and hypoxia, the expression of microRNA-125 was observed to be regulated in hypoxia by PTEN rather than by HIF-1 (FIG. 3).

In addition, the modulation of PTEN was found to lead to a significant increase in VEGF level in hypoxic conditions and the cells in hypoxia were measured to decrease the increased VEGF level to a normal one when treated with microRNA-125 (FIG. 3), indicating together that microRNA-125 can suppress the angiogenesis induced by the inactivation of PTEN.

The anticancer activity of microRNA-125 was also confirmed in terms of the inhibitory activity against the invasion of cervical cancer cells as well as brain cancer cells. (FIGS. 4 and 8).

microRNA-125 was also in vivo assayed for anticancer activity. Mice transplanted with microRNA-125-expressing cell lines survived longer compared to non-transplanted controls and did not lose weight (FIG. 5).

Consequently, the inactivation of PTEN in hypoxic brain cancer cells brings about a reduction in microRNA-125 expression level and thus frees VEGF expression from the detention of microRNA-125, leading to an increase in angiogenesis. Thus, microRNA-125 functions as an important factor in restraining the incidence of brain cancer. Its elevated expression levels suppress the hypoxia-induced angiogenesis of cancer cells, thus constraining cancer from invasion and metastasis.

In accordance with an embodiment of the present invention, the anticancer composition comprises an expression vector carrying the microRNA-125 (mir-125).

The microRNA-125 nucleic acid molecules of the present invention can be introduced into cells using DEAE-dextran-, nucleoprotein- or liposome-mediated DNA transfection. In this regard, the microRNA-125 nucleic acid molecules may be anchored at a carrier allowing for the effective delivery of nucleic acid molecules into cells. Preferably, the carrier is a vector, whether viral or non-viral. Examples of viral vectors useful in the present invention include vectors derived from lentivirus, retrovirus, adenovirus, herpes virus and avipox virus, preferably from lentivirus, but are not limited thereto. Lentivirus, a kind of retrovirus, can productively infect both dividing and non-dividing cells because its pre-integration complex (virus “shell”) can get through the nucleopores or intact membrane of the nucleus of the target cell.

In a preferred embodiment, the vector carrying the microRNA-125 nucleic acid molecule further anchors a selection marker therein. As used herein, the term “selection marker” is intended to mean a marker for readily selecting a cell to which the microRNA-125 nucleic acid molecule is introduced. No particular limitations are imparted to the marker provided that it enables the introduction of the vector to be readily detected or measured. Typically, markers for conferring on transformants selectable phenotypes such as drug resistance, autotrophy, resistance to cytotoxic agents, or surface protein expression. Examples of the markers include green fluorescent protein (GFP), puromycin, neomycin (Neo), hygromycin (Hyg), histidinol dehydrogenase gene (hisD) and guanine phosphosribosyltransferase (Gpt), with preference for GFP and puromycin.

In accordance with another embodiment of the present invention, the anticancer composition comprises a cell into which the microRNA-125 (mir-125) nucleic acid molecule is introduced.

The term “introduction”, as used herein, is intended to mean the delivery of foreign DNA into cells through transfection or transduction. Transfection can be carried out using various methods well known in the art, including calcium phosphate-DNA co-precipitation, DEAE-dextran-mediated transfection, polybrene-mediated transfection, electroporation, microinjection, liposome fusion, Lipofectamine transfection, and protoplast fusion. Transduction refers to a process whereby foreign DNA is transferred to another cell via a virus or viral vector on the basis of infection.

When transplanted into cancer tissues, the cells with microRNA-125 nucleic acid molecules introduced thereinto can suppress the invasion and metastasis of cancer due to the high expression level of microRNA-125. Hence, a composition comprising a cell into which the microRNA-125 nucleic acid molecule is introduced may be used as a therapeutic for cancer.

In an embodiment, the anticancer composition comprising microRNA-125 nucleic acid molecules in accordance with the present invention may further comprise and be formulated with a pharmaceutically acceptable vehicle. The term “pharmaceutically acceptable vehicle”, as used herein, is intended to refer to a carrier or a diluent which does not destroy the pharmaceutical activities and properties of the ingredient without the irritation of the subject to be treated. For use in liquid formulations of the composition of the present invention, the pharmaceutically acceptable vehicle is preferably suitable for sterilization and living body. The active ingredient of the present invention may be formulated with one selected from among saline, sterile water, Ringer's solution, buffered saline, albumin injection, dextrose solution, maltodextrose solution, glycerol, ethanol and combinations thereof, and if necessary, in combination with another conventional additives including antioxidants, buffer, bacteriostatic agents, etc. Alternatively, the composition of the present invention may be formulated into injections, pills, capsules, granules, or tablets with diluents, dispersants, surfactants, binders and/or lubricants.

The anticancer composition comprising a microRNA-125 nucleic acid molecule and a pharmaceutically acceptable vehicle in accordance with the present invention may be formulated into any dosage form, whether oral or non-oral. The pharmaceutical formulations according to the present invention may be administered via oral, rectal, nasal, topical (including bolus and sublingual), transdermal, vaginal, or parenteral (including intramuscular, subcutaneous and intravenous) routes or by inhalation or insufflation.

Examples of the oral dosage forms formulated with the composition of the present invention include tablets, troches, lozenges, water-soluble or oil suspensions, powders, granules, emulsions, hard or soft capsules, syrups or elixirs. For tablet or capsule formulations, useful are additives including a binder, such as lactose, saccharose, sorbitol, mannitol, starch, amylopectin, cellulose or gelatin, an excipent such as dicalcium phosphate, a disintegrant such as corn starch or sweet potato starch, and an lubricant, such as magnesium stearate, calcium stearate, sodium stearyl fumarate, sodium or polyethylene glycol. In addition to these additives, a liquid carrier such as fat oil may be used for capsule formulations.

For use in parenteral administration, the composition of the present invention may be formulated into injections via subcutaneous, intravenous or intramuscular routes, suppositories, or sprays via inhalation, such as aerosols. Injections may be prepared by mixing the composition of the present invention with a stabilizer or buffer in water to give solutions or suspensions which are packaged in unit dosages such as ampules or vials. For suppositories, the composition of the present invention may be formulated with a conventional base such as cocoa butter or glyceride, or an enema. The composition of the present invention in the form of a water-dispersed concentrate or a wet powder may be formulated with a propellant to prepare an aerosol spray.

In accordance with another aspect thereof, the present invention pertains to a method for the treatment of cancers characterized by a low expression level of microRNA-125, comprising the administration of the anticancer composition comprising a microRNA-125 nucleic acid molecule.

The term “administration”, as used herein, is intended to mean the introduction of the pharmaceutical composition of the present invention to a subject using any appropriate method, as exemplified by the delivery of the microRNA-125 nucleic acid molecule using viral or non-viral technology or by the transplantation of cells expressing microRNA-125. As long as it ensures the arrival of the composition of the present invention to a tissue of interest, any route may be taken for administration. For example, the composition of the present invention may be administered orally, rectally, topically, intravenously, intraperitoneally, intramuscularly, intraarterially, transdermally, intranasally, intrathoracically, intraocularly, or intradermally. Preferably, the anticancer composition of the present invention may be administered locally into cancer tissues.

The treatment method of the present invention includes administering the anticancer composition of the present invention in a pharmaceutically effective amount. It will be apparent to those skilled in the art that the suitable total daily dose may be determined by an attending physician within the scope of sound medical judgment. The specific therapeutically effective dose level for any particular patient may vary depending on a variety of factors, including the kind and degree of desired reaction, the specific composition, including the use of any other agents according to the intended use, the patient's age, weight, general health, gender, and diet, the time of administration, route of administration, and rate of the excretion of the composition; the duration of the treatment; other drugs used in combination or coincidentally with the specific composition; and like factors well known in the medical arts. Accordingly, the effective amount of the anticancer composition suitable for the purpose of the present invention is preferably determined in full consideration of the above-mentioned factors. In some case, the anticancer composition of the present invention may also be administered in combination with well-known anticancer agents so as to afford increased anticancer effects.

Further, the treatment method of the present invention may be applied to any animal suffering from cancer characterized by a low expression level of microRNA-125. Examples of the animal include cows, pigs, sheep, horses, dogs and cats as well as humans and primates.

In accordance with another aspect thereof, the present invention pertains to a method for suppressing the invasion and metastasis of cancer cells using the microRNA-125 nucleic acid molecule.

In accordance with another aspect thereof, the present invention pertains to a method for inhibiting hypoxia-induced angiogenesis. When expressed in cancer cells, the microRNA-125 nucleic acid molecule of the present invention can suppress the secretion of VEGF (vascular endothelial growth factor) to inhibit angiogenesis therein, which thus leads to the inhibition of invasion and metastasis of cancer cells. Particularly, the microRNA-125 nucleic acid molecule of the present invention is effective in suppressing the angiogenesis induced by the inactivation of PTEN (phosphatase and tensin homolog deleted on chromosome ten) rather than the angiogenesis induced in a hypoxia by HIF-1, implying that the expression of microRNA-125 in cells is regulated by PTEN. Consequently, when the expression of PTEN is increased in hypoxia, the expression of microRNA-125 is also increased to suppress angiogenesis and thus to prevent the invasion and metastasis of cancer cells.

For the treatment of cancer cells with the microRNA-125 nucleic acid molecules of the present invention, viral or non-viral delivery systems may be employed. Examples of the viral delivery systems include vectors derived from lentivirus, retrovirus, adenovirus, herpes virus and avipox virus, but are not limited thereto. Useful in the non-viral delivery systems are lipid-mediated transfection, liposomes, immunoliposomes, lipofectin, anionic surface amphiphiles, and combinations thereof.

In accordance with another aspect thereof, the present invention pertains to a marker composition for the diagnosis of brain cancer and brain tumor, comprising an agent for measuring a microRNA-125 expression level, and a diagnostic kit comprising the same.

As used herein, the term “agent for measuring a microRNA-125 expression level” is intended to refer to a molecule which is reacted with microRNA-125 to determine the expression level of microRNA-125. As the agent for measuring a microRNA-125 expression level, a primer or probe specific microRNA-125 is useful. The expression level of microRNA-125 may be determined using PCR with the primer or hybridization with the probe. The diagnostic kit based on the marker composition may contain various tools and reagents known to be useful in detection, such as appropriate carriers, detectable signal-generating labels, solubilizers, cleansers, buffers, stabilizers, etc.

In accordance with another aspect thereof, the present invention pertains to a method for screening a compound therapeutic for brain cancer or brain tumors, using the microRNA-125 nucleic acid molecule of the present invention. It comprises treating brain cancer or tumor cells with a microRNA-125 (mir-125)-regulating candidate; and measuring an expression level of microRNA-125 in a hypoxic condition. After the treatment of brain cancer or tumor cells with therapeutic candidates, microRNA-125 expression levels in the cells are measured such that ones which induce an increase in microRNA-125 expression level are selected as being useful in the treatment of microRNA-125-mediated cancers.

### Advantageous Effects

Showing the ability to inhibit angiogenesis through the suppression of hypoxia-induced VEGF secretion in cancer cells, the anticancer composition comprising a microRNA-125 nucleic acid molecule in accordance with the present invention is effectively inhibitory of the invasion and metastasis of cancer cells and useful as gene therapy for various cancers.

### DESCRIPTION OF DRAWINGS

FIG. 1 shows a microarray assay for microRNA expression patterns in hypoxia and normoxia. A) a schematic view illustrating the selection of microRNAs specific for hypoxia. MicroRNAs are isolated from the astrocytes of brain cancer cells which have been cultured in a normal or hypoxic state, followed by comparison of the microRNAs. B) microarray assay results showing expression patterns of microRNAs in various brain cancer cell lines.

FIG. 2 is a graph showing VEGF secretion levels in the presence of various microRNAs under a normoxia and a hypoxic condition.

FIG. 3 provides various results illustrating the control mechanism of miRNA125 in cells. A) secretion levels of VEGF in the presence of siRNA against HIF-1, B) expression levels of miRNA125 in the presence of siRNA against HIF-1, C) Western blots showing expression patterns of HIF-1 protein in the presence of siHIF and mir-125a, D) expression patterns of VEGF in hypoxia upon the inactivation of PTEN, E) secretion levels of VEGF in PTEN-inactivated cells treated with PTEN alone and in combination with antagonists of microRNA-125.

FIG. 4 shows the inhibitory activity of mir125 against the invasion of brain cancer cells in a graph and photographs.

FIG. 5 shows experimental data for anticancer effects of mir125 in mice implanted with mir125-expressing cells. A) mouse model, B) schematic diagram illustrating a process of infusing brain cancer cells into the brain, C) survival of brain cancer-induced mice when treated with mir125-expressing cells, D) body weights of brain cancer-induced mice when treated with mir125-expressing cells.

FIG. 6 shows nucleotide sequences and positions on genome of precursor mir125 and mature mir125. Nucleotide sequences and positions on human genome of miR-125 refer to NCBI GeneID Nos. mir125a (406910), mir125b1 (406911) and mir125b2 (406912), and miRBASE (http://microrna.sanger.ac.uk/) accession numbers mir125a (MI0000469), mir125b1 (MI0000446) and mir125b2 (MI0000470).

FIG. 7 is a schematic diagram showing the structure of lentivirus for use in the expression of microRNA-125.

FIG. 8 shows the inhibitory activity of mir125 against the invasion of the cervical cancer cell line HeLa in photographs and a histogram.

### MODE FOR INVENTION

A better understanding of the present invention may be obtained through the following examples which are set forth to illustrate, but are not to be construed as limiting the present invention.

### EXAMPLE 1

### Cell Culture

The normal primary cell astrocyte and various brain tumor cells (LN-18 (CRL-2610), U-87 MG (HTB-14), LN-229 (CRL-2611), U251, U373 and LN428) were cultured in DMEM/high glucose (4500 mg/L) media (Hyclone) supplemented with 10% FBS (fetal bovine serum) in an incubator with 5% CO₂/21% O₂ atmosphere. For incubation in a hypoxic condition, the oxygen concentration was decreased to 1%. The HeLa cell line, derived from cervical cancer cells, was also cultured in the same manner.

### EXAMPLE 2

### Transient Transfection

miRNAs (microRNAs), siRNAs and vectors carrying genes of interest were transfected into cells using electroporation with Cell Line Nucleofector kit T solution (Amaxa). For this purpose, first, cells were counted using a hematocytometer and aliquoted at a population of 1.5×10⁶ cells/tube into 1.5 mL E-tubes, followed by centrifugation at 1000 rpm for 3 min to harvest cells. They were washed once with DPBS and mixed with 100 μl of T solution in combination with 2 μg DNA or 100 nM siRNA or microRNA before electroporation using an Amaxa electroporator. Immediately after the electroporation, the cells were mixed with 1 ml of DMEM supplemented with 10% FBS, transferred into 100 mm dishes and supplemented with 10 ml of the medium. Then, the dishes were incubated for 48 hrs in a cell incubator before the next experiment.

### EXAMPLE 3

### Comparison of microRNA Expression Patterns through Microarray Assay

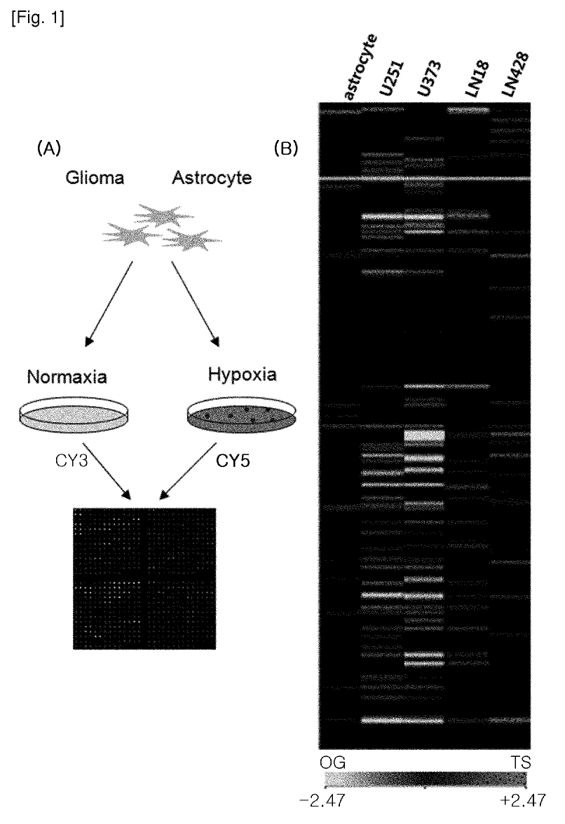

A microarray assay was conducted to compare microRNA expression patterns in various cell lines under hypoxic and normoxic conditions (FIG. 1). Following incubation for 24 hrs in normoxia or hypoxia (1% O₂), the normal brain cell astrocyte and various brain tumor cells were subjected to RNA isolation with Triozol (Invitrogen). The RNAs thus isolated were analyzed for miRNA expression levels under each condition by E-biogen Inc. using Agilent Microarray (Agilent technology, USA).

### EXAMPLE 4

### Regulation of Hypoxia-Induced VEGF Secretion by Various microRNAs

The miRNAs which were found to change in expression level between hypoxia and normoxia as assayed by Microarray of Example 3 were ordered from Ambion, U.S.A. They were transfected into the brain cancer cell line U373 through electroporation (electroporator, Amaxa) and incubated for 48 hrs and then for an additional 24 hrs under normoxia and hypoxia. The medium supernatants were obtained and assayed for VEGF secretion levels using ELISA.

For VEGF ELISA assay, human VEGF QuantiGlo ELISA Kit (R&D Systems) was used. In detail, the Assay Diluent was added in an amount of 150 μl to each well of the kit, along with 50 μl of a standard solution and 50 μl of a culture medium sample to be assayed for VEGF level, and incubated at room temperature for 2 hrs with shaking. The liquids were aspirated from each well which was then washed four times. 200 μl of a conjugator was added to each well and incubated at room temperature for three hrs with shaking. Again, the liquid was aspirated before four washings. Each well was incubated for 20 min with 100 μl of Working Glo Reagent in the absence of light. In this regard, it was wrapped with foil and incubated in a dark room. Following the completion of reaction, VEGF levels were determined using a luminometer.

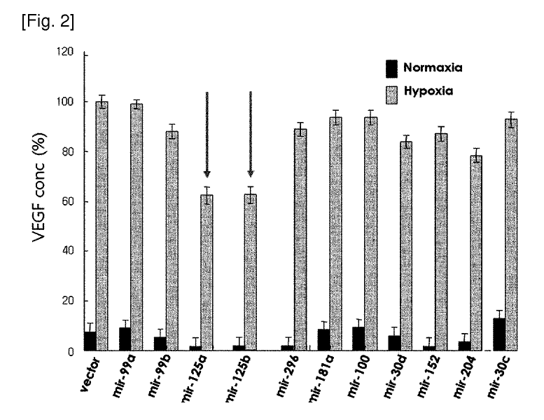

After the examination of the effects of various microRNAs on VEGF secretion in normoxia and hypoxia, it was found that both mir-125a and mir-125b inhibit hypoxia-induced VEGF secretion (FIG. 2).

### EXAMPLE 5

### Production of Lentivirus and Construction of a Cell Line Expressing microRNA

A vector which carries microRNA 125a and 125b and allows for the production of a Lentivirus having the structure of FIG. 7 was constructed. The resulting vector (3 μg) was used in combination with ViraPower™ Packaging Mix (9 μg, Invitrogen) which contains pLP/VSVG, pLP1 and pLP2, each encoding viral structural proteins, together, to produce the virus. For use as a host for producing the virus, 293FT cells were cultured in a suitable culture medium prepared according to the manufacturer's protocol. Transfection was conducted with Fugene6 (Roche) according to the manufacturer's protocol. The transfected 293FT cells were further cultured for 48 hrs and progeny viruses were collected from the medium supernatant.

Viral titers were determined by FACS taking advantage of GFP which was anchored at the vector. In this regard, serial dilutions of from 10⁻¹ to 10⁻⁹ of the collected viruses were added in an amount of 1 ml per well to U373 cells previously aliquoted into 6-well plates. 48 Hours after the addition, the cells were separated with trypsin-EDTA and washed with DPBS. Among them, GFP-expressing cells were counted using FACS and used to determine viral titers as percentages thereof. U373 cells were infected at an MOI of 10 TU (transducing unit)/cell with the tittered virus to construct U373 cell lines which transcribed miR-125a and miR-125b, respectively.

### EXAMPLE 6

### Examination of Regulatory Mechanism of microRNA-125

In order to determine the mechanism by which microRNA-125 is controlled in cells, the transcription factor HIF-1 (hypoxia inducible factor 1) and the tumor suppressor gene PTEN, both known to play important roles in VEGF expression, were examined for relationship with microRNA-125. For use as microRNA antagonists to study miRNA functions, nucleotides were designed to specifically bind to miRNAs within cells. For this experiment, microRNA antagonists were purchased from Dharmacon. The lentivirus vector according to Example 5 was used as an miRNA 125 expression vector. Both miRNA and siRNA were used at a concentration of 100 nM for infection into the cells, respectively.

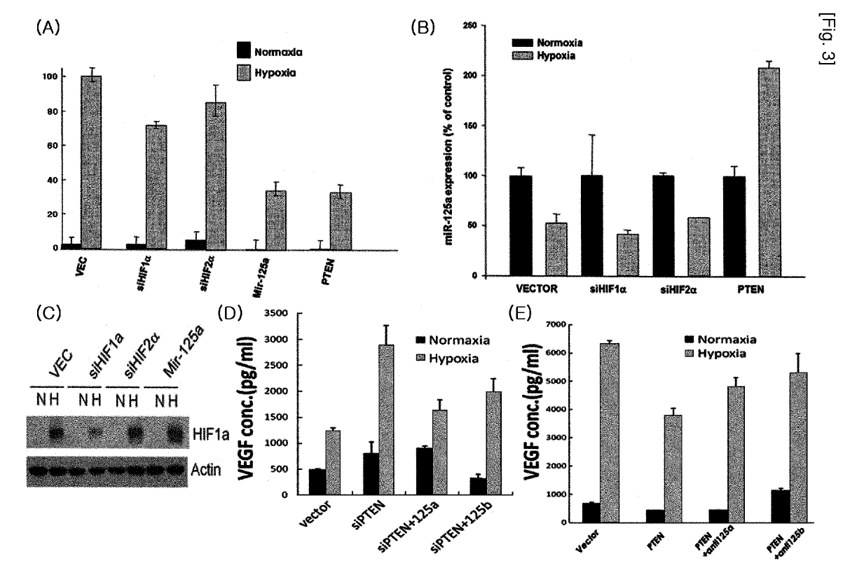

In order to examine the control of angiogenesis by HIF-1 inhibition, PTEN expression and microRNA-125 expression, U373 cells were respectively transfected with siHIF-1, siHIF-2, PTEN and mir-125a by electroporation and cultured for 48 hrs. After the incubation of the transfected cells for an additional 24 hrs in normoxia and hypoxia, cell medium supernatants were assayed for VEGF excretion levels using an ELISA kit (R&D System) (FIG. 3A).

As is apparent from the data of FIG. 3A, VEGF secretion was suppressed when HIF-1 was inhibited by siHIF-1 in hypoxia or when PTEN or microRNA-125 was expressed. Noteworthily, the expression of microRNA-125 or PTEN resulted in the suppression of VEGF secretion to a higher extent than did the inhibition of HIF-1 activity.

An examination was made of the effect of HIF-1 inhibition or PTEN expression on microRNA-125 expression in normoxia and hypoxia. For this purpose, U373 cells were respectively transfected with siHIF, PTEN and mir-125a by electroporation and cultured for 48 hrs. After incubation for an additional 24 hrs in normoxia and hypoxia, the cells were subjected to RNA isolation using Trizol (Invitrogen, USA). The RNA thus obtained was amplified by real-time PCR to determine mir-125a expression levels in each condition (FIG. 3B).

As understood in the graph of FIG. 3B, the microRNA-125 expression level in hypoxia was increased with PTEN expression rather than HIF-1 inhibition, implying that microRNA-125 expression can be controlled by PTEN expression.

In addition, the effects of siHIF and mir-125a on the translation of HIF-1 were examined through Western blot assay. After electroporation with siHIF and mir-125a, respectively, U373 cells were incubated for 48 hrs in normoxia and then for an additional 24 hrs in normoxia and hypoxia. Proteins were isolated from the cells and assayed for HIF-1a expression level using Western blotting. As seen in the expression patterns of FIG. 3c, HIF-1a expression was greatly reduced by siHIF-1a.

In hypoxia, VEGF expression patterns were also examined when PTEN was modulated and when microRNA-125 was overexpressed while PTEN was modulated. In greater detail, after being infected with siPTEN-expressing lentivirus to inactivate PTEN therein, LN229 cells were additionally infected with mir-125a or mir-125b lentiviruses to construct stable cell lines which expressed mir-125a and mir-125b, respectively. Using an ELISA kit, VEGF expression levels were measured in the cells wherein PTEN was modulated to inactivation and wherein mir-125a or mir-125b was overexpressed while PTEN was inactivated (FIG. 3D).

As seen in FIG. 3D, PTEN modulation caused an increase in VEGF expression level under a hypoxic condition, such that an increased secretion level of VEGF was detected in the medium. Also, the overexpression of microRNA-125 in the cells of which the VEGF expression was increased by PTEN modulation was observed to decrease the elevated VEGF expression levels, indicating that microRNA-125 can regulate the angiogenesis resulting from PTEN inactivation.

In addition, an examination was made of whether the inhibition of VEGF secretion by PTEN is mediated by microRNA-125 or not in hypoxia. To this end, VEGF secretion was measured from PTEN-modulated cells when PTEN was added into them. On the other hand, when the cells were treated with PTEN in combination with microRNA-125 antagomirs, anti125a or anti125b, the secreted VEGF concentrations were compared (FIG. 3E). For example, after a U373 cell line in which PTEN was inactivated by mutation was transfected with PTEN by electroporation, the concentration of the VEGF secreted into the medium was measured. Also, VEFG expression levels were measured using an ELISA kit after PTEN was introduced in combination with the antagonists of mir-125a and mir-125b into the cell line.

As seen in FIG. 3E, the introduction of PTEN into the cells with inactivated PTEN was found to induce the suppression of VEGF expression in hypoxia. Also, when the cells were treated with PTEN in combination with a microRNA-125 antogomir, the VEGF expression was inhibited to lesser extent than when the cells were treated with PTEN alone. Accordingly, the data of FIG. 3E show that the inhibition of hypoxia-induced angiogenesis by PTEN is mediated through microRNA-125.

Collectively, the results obtained above suggest that in brain cancer cells with inactivated PTEN, microRNA-125 expression level is decreased thereby to increase VEGF expression, leading to hypoxia-induced angiogenesis and that microRNA-125 plays an important role in brain cancer.

### EXAMPLE 7

### Influence of microRNA-125 on Cancer Cell Invasion

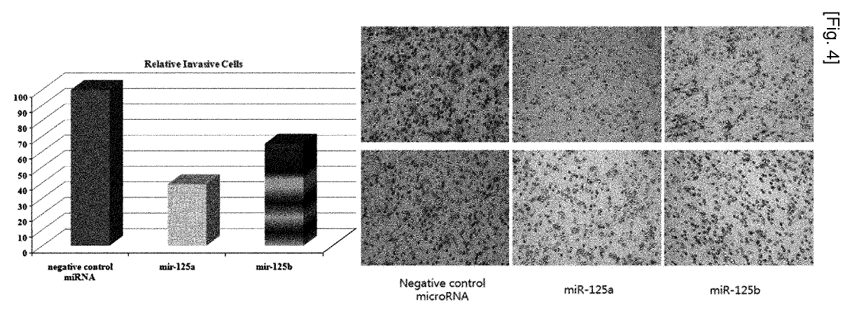

In order to examine the influence of microRNA-125 on the invasion of brain cancer cells, mir-125a, mir125b and negative control miRNA (Dharmacon) were introduced into respective U373 cells (FIG. 4). In detail, cells were counted one day before the experiment and mir-125a, mir125b and a negative control miRNA were respectively electroporated into 1.5×10⁶ cells which were then incubated for 48 hrs. Matrigel (growth factor reduced BD Matrigel matrix) was put into an upper chamber of a 24-well transwell for invasion assay. The cells were suspended in serum-free DMEM media and the cell suspension was put at a density of 1.5×10⁶ cells/well onto the matrigel in the upper chamber of the transwell. A DMEM medium containing 1% BSA was filled in the lower chamber of the transwell, followed by incubating the transwell at 37° C. for 24 hrs in a CO₂ incubator. Subsequently, the cells were fixed and stained with a Diff-Quick kit (Sysmex) over both cytoplasm and nucleus. Non-invaded cells on the top of the transwell were scraped off with a cotton swab and the invaded cells, which penetrated into the transwell, were counted.

The data of FIG. 4 shows that mir125a and mir125b reduced the invasion by about 60% and about 40% respectively, as compared to the negative control.

### EXAMPLE 8

### Anticancer Activity of microRNA-125

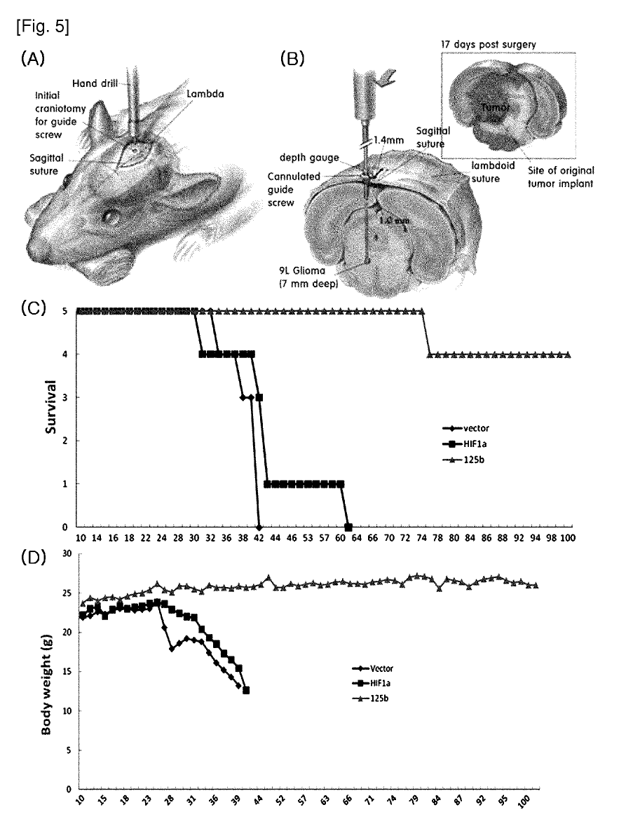

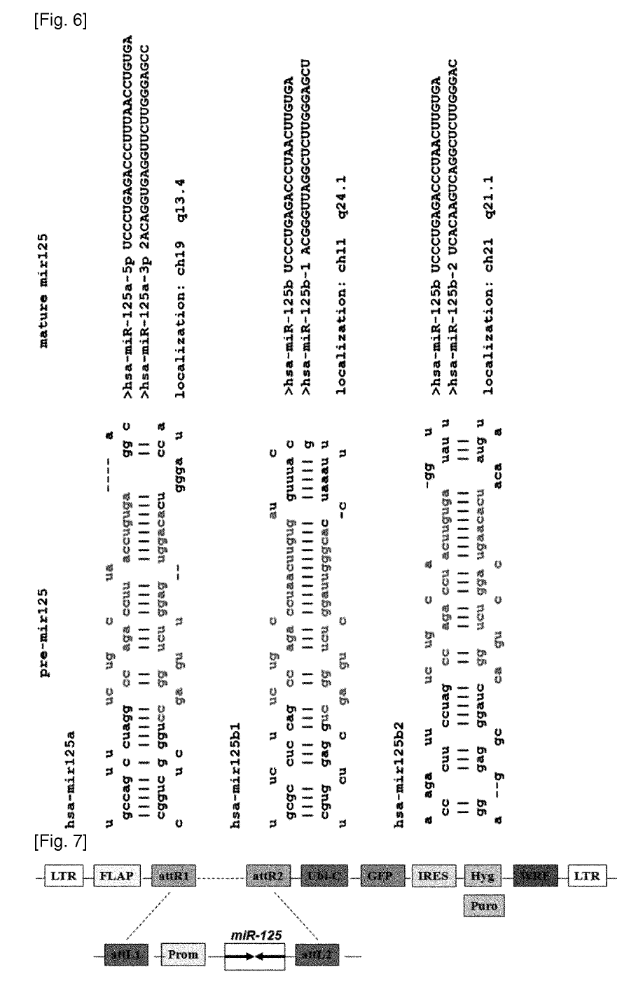

In order to examine whether miR-125b and HIF1a can regulate cancer, cell lines producing miR-125b and HIF1a were constructed and transplanted into mice, followed by measuring the mice for survival and body weight (FIG. 5).

In greater detail, U373-MG cells were infected with Lentivirus carrying miR-125b, HIF1a or a blank vector to construct stable cell lines which produced miR-125b or HIF1a. A guide-screw (Plastics one) was fixed at a position 1 mm left lateral to and 2 mm inferior to the bregma of the skull of each of 15 female nude mice (Balb-C/nu-nu, Halan) 6 weeks old. A dummy (Plastics One) was inserted into each guide. About 10 days later, the dummy was replaced with a microinjection cannula (Hamilton) by which 5×10⁵ cells of each of the constructed cell lines were injected into the caudate putamen to a depth of 4 mm. From day 10 after the dummy was reinserted into the guide-screw, the mice were monitored for changes in body weight, behavior and health state for about 120 days.

As seen in FIG. 5, the control mice suffered from weight loss and died in about 40 days while the mice into which the mir-125b-expressing cell line was transplanted lived for about 120 days with relatively constant body weight, indicating that mir-125b exhibits anticancer activities in animal tissues sufficiently to inhibit cancer growth.

### EXAMPLE 9

### Influence of microRNA-125 on Invasion of Cervical Cancer Cells

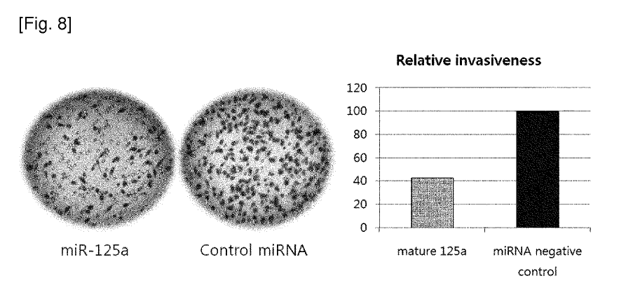

In order to examine the effect of mir-125 on cancer cells other than brain cancer cells, the same invasion assay as in Example 7 was performed with the cervical cancer cell line HeLa (FIG. 8). In detail, cells were counted one day before the experiment and mir-125a or a negative control miRNA (Dharmacon) was electroporated into 1.5×10⁶ cells of HeLa which were then incubated for 48 hrs. Matrigel (growth factor reduced BD Matrigel matrix) was put into an upper chamber of a 24-well transwell for invasion assay. The cells were suspended in serum-free DMEM media and the cell suspension was put at a density of 1.5×10⁵ cells/well onto the matrigel in the upper chamber of the transwell. A DMEM medium containing 1% BSA was filled in the lower chamber of the transwell, followed by incubating the transwell at 37° C. for 24 hrs in a CO₂ incubator. Subsequently, the cells were fixed and stained with a Diff-Quick kit (Sysmex) over both cytoplasm and nucleus. Non-invaded cells on the top of the transwell were scraped off with a cotton swab and the invaded cells, which penetrated into the transwell, were counted.

The data of FIG. 8 shows that mir125a significantly reduced the invasion, as compared to the negative control, indicating that microRNA-125 has inhibitory activity against cervical cancer as well as brain cancer by suppressing the invasion of both types of cells.

### INDUSTRIAL APPLICABILITY

Found to inhibit hypoxia-induced angiogenesis of cancer cells and suppress the growth, invasion and metastasis of cancer cells, as described hereinbefore, the anticancer composition comprising microRNA-125 in accordance with the present invention can be useful in gene therapy effective for various cancers, particularly ones which are difficult to treat with surgery, chemical therapy or radiation therapy.

1.-20. (canceled)

21. A composition for treatment of brain cancer or cervical cancer, comprising microRNA-125a.

22. A composition for treatment of brain cancer, cervical cancer, angioma, angiofibroma, arteriosclerosis, vascular adhesion, scleroderma, neovascular glaucoma, diabetic retinopathy, neovascular corneal disorders, arthritis, psoriasis, telangectasia, pyogenic granuloma, or Alzheimer's disease, comprising microRNA-125b1 or microRNA-125b2.

23. The composition according to claim 21 or 22, wherein the cancer is low in microRNA-125 expression level.

24. The composition according to claim 21 or 22, having inhibitory activity against invasion and metastasis of cancer cells.

25. The composition according to claim 21 or 22, having inhibitory activity against hypoxia-induced angiogenesis.

26. The composition according to claim 21 or 22, wherein the microRNA-125a, microRNA-125b1 or microRNA-125b2 nucleic acid molecule is present in an expression vector.

27. The composition according to claim 26, wherein the vector is a viral vector derived from a group consisting of lentivirus, retrovirus, adenovirus, herpesvirus and avipoxvirus.

28. The composition according to claim 21 or 22, wherein the microRNA-125a, microRNA-125b1 or microRNA-125b2 nucleic acid molecule is introduced into a cell.

29. The composition according to claim 21 or 22, further comprising a pharmaceutically acceptable vehicle.

30. A marker composition for diagnosis of brain cancer or brain tumor, comprising an agent for measuring a microRNA-125a, microRNA-125b1 or microRNA-125b2 expression level.

31. A method for treating brain cancer or cervical cancer, comprising administering the composition of claim 21, wherein the cancer is low in microRNA-125a expression level.

32. A method for treating brain cancer or cervical cancer, comprising administering the composition of claim 22, wherein the cancer is low in microRNA-125b1 or microRNA-125b2 expression level.

33. A method for treatment of a hypoxia-induced angiogenesis-associated disease, comprising administering the composition of claim 22.

34. The method according to claim 33, wherein the treatment comprises the step of suppressing secretion of VEGF (vascular endothelial growth factor).

35. The method according to claim 33, wherein the treatment comprises the step of suppressing the angiogenesis induced by the inactivation of PTEN (phosphatase and tensin homolog deleted on chromosome ten).


SEQUENCE LISTING (5 sequences)

SEQ ID NO: 1 - RNA, 24 nt, Homo sapiens
  mature sequence of hsa-miR-125a-5p
ucccugagacccuuuaaccuguga

SEQ ID NO: 2 - RNA, 22 nt, Homo sapiens
  mature sequence of hsa-miR-125a-3p
acaggugagguucuugggagcc

SEQ ID NO: 3 - RNA, 22 nt, Homo sapiens
  mature sequence of hsa-miR-125b
ucccugagacccuaacuuguga

SEQ ID NO: 4 - RNA, 22 nt, Homo sapiens
  mature sequence of hsa-miR-125b-1
acggguuaggcucuugggagcu

SEQ ID NO: 5 - RNA, 22 nt, Homo sapiens
  mature sequence of hsa-miR-125b-2
ucacaagucaggcucuugggac